SEQUENCE LISTING (3 sequences)

SEQ ID NO: 1 - protein, 93 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKRFKM

SEQ ID NO: 2 - protein, 211 aa, Homo sapiens [sequence omitted]

SEQ ID NO: 3 - protein, 622 aa, Homo sapiens
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

(19) United States
(12) Patent Application Publication
(10) Pub. No.: US 2024/0139376 A1
(43) Pub. Date: May 2, 2024

(54) RECONSTITUTION OF EXTRACELLULAR MATRIXES FOR MUSCULOSKELETAL JOINT TISSUE REPAIR USING BIOMIMETIC BIOLOGIC AND SYNTHETIC FACTORS

(71) Applicant: Rhode Island Hospital, Providence, RI (US)

(72) Inventors: Chathuraka Teekshana JAYASURIYA, Barrington, RI (US); Daniel S. YANG, Irvine, CA (US)

(21) Appl. No.: 18/276,593

(22) PCT Filed: Feb. 11, 2022
(86) PCT No.: PCT/US2022/016171

§ 371 Date: Aug. 9, 2023

Related U.S. Application Data
(60) Provisional application No. 63/148,614, filed on Feb. 12, 2021.

(51) Int. Cl. A61L 27/38; A61L 27/22; A61L 27/54

(52) U.S. Cl. CPC A61L 27/3817; A61L 27/225; A61L 27/3895; A61L 27/54; A61L 2400/06; A61L 2430/30

(57) ABSTRACT

The invention features compositions and methods for repairing musculoskeletal defects or injuries using a bioactive scaffold comprising fibrinogen, thrombin, SDF-1 and/or KGN as well as methods of making the scaffold.

### RELATED APPLICATIONS

This application claims the benefit of priority under 35 U.S.C. § 119(e) to U.S. Provisional Application No. 63/148,614, filed Feb. 12, 2021, the entire contents of which is incorporated herein by reference in its entirety.

### STATEMENT AS TO FEDERALLY SPONSORED RESEARCH

This invention was made with government support under R21AR077326 awarded by the National Institutes of Health and W81XWH-2010773 awarded by the Department of Defense. The government has certain rights in the invention.

### TECHNICAL FIELD

The current subject matter relates to methods and systems for treating musculoskeletal injuries or disorders.

### BACKGROUND

Musculoskeletal conditions comprise more than 150 conditions that affect the locomotor system of individuals. They range from those that arise suddenly and are short-lived, such as fractures, sprains and strains, to lifelong conditions associated with ongoing functioning limitations and disability.

Musculoskeletal conditions are typically characterized by pain (often persistent) and limitations in mobility, dexterity and overall level of functioning, reducing people's ability to work. Although surgical techniques, physical therapy, biomaterials, and muscular tissue engineering as well as cell therapy presently exist, there is a great need to develop methods and materials, which promote skeletal muscle repair and functional regeneration.

### SUMMARY OF THE INVENTION

The invention provides a solution to the drawbacks and problems associated with existing methods of treatment which promote skeletal muscle repair and functional regeneration. Accordingly, the invention features a bioactive scaffold comprising fibrinogen, thrombin, stromal cell derived factor 1 (SDF-1), and kartogenin (KGN) to form a chondrogenic fibrin glue into which a homogenous solution of skeletal tissue derived mesenchymal progenitor cells (STMSCs) is encapsulated. For example, components may include: fibrinogen (GENBANK: CAA50740); thrombin (GENBANK: NP_000497); and/or SDF-1 (GENBANK: P48061.1).

For example, the STMSCs comprise cartilage-derived mesenchymal progenitor cells (CPCs). These cells are multipotent progenitor cells that are found in healthy non-arthritic articular cartilage tissues (Jayasuriya et al. 2019 January; 37(1):102-114. Epub 2018 Nov. 2. PubMed PMID: 30358021; PubMed Central PMCID: PMC6312732).

In aspects, provided herein is a bioactive scaffold comprising a chondrogenic fibrin glue into which a population of skeletal tissue derived mesenchymal progenitor cells (STMSCs) is encapsulated. For example, the population of cells include a suspension of cells in a solution. In embodiments, the chondrogenic fibrin glue includes fibrinogen, thrombin, stromal cell derived factor 1 (SDF-1), and kartogenin (KGN). In embodiments, the fibrin glue comprises skeletal tissue derived mesenchymal progenitor cells (STMSCs). In embodiments, the fibrin glue includes cartilage-derived mesenchymal progenitor cells (CPCs). In embodiments, the CPCs are cells deposited with ATCC accession number PTA-127250.

A method of repairing a musculoskeletal tissue defect or injury in a mammalian subject, comprising contacting the defect or site of injury with the bioactive scaffold described above is also within the invention. For example, the scaffold is administered to the subject by injection or materialized to form a gel first, then placed at the injury site arthroscopically through a portal. After the initial injection (and/or placement) of the gel at the injury site, it is anticipated that further cells injected into the joint (as therapy) will chemotactically migrate to the scaffold due to the presence of SDF-1. Hence, this will help the subsequently injected cells to make their way to the injury site to further accelerate healing. Injected cells make their way to the injury site to further accelerate healing. The subject is a mammal such as a human or companion animal such as a dog or cat. The methods are also useful to treat performance animal, e.g., race horses. For example, the invention is also applicable to other animals, such as livestock, bovine, chickens, goats, sheep or pigs.

In examples, the bioactive scaffold refers to the combination of fibrinogen, thrombin, SDF-1, KGN and CPCs. In other examples, the gel refers to the bioactive scaffold after it has solidified (used interchangeably herein with “glue”).

The invention also encompasses methods of manufacturing a bioactive scaffold.

The scaffold is created by combining two components. Component #1 is thrombin (350-700 units/mL), calcium chloride (30-50 μm/mL), SDF-1 (30 ng/mL), KGN (0.1 μg/mL) and cells in distilled water. Component #2 is fibrinogen (60-120 mg/mL) and the synthetic crosslinker aprotinin (2250-3750 KIU/mL) in distilled water. Component #1 and #2 are combined in a 1:1 ration by volume. 4×10⁶ cells can be embedded per 10 μL of the gel (or bioactive scaffold) using this methodology of bioactive scaffold preparation described (FIG. 1). Re-suspended thrombin is measured in units per mL, see, e.g., Hemker, H. C., Handbook of Synthetic Substrates for the Coagulation and Fibrinolytic System, Martinus Nijhoff (Boston, MA)/Springer (Dordrecht, The Netherlands), pp. 95-101 (1983) and Biggs, R., ed., Human Blood Coagulation, Haemostasis and Thrombosis (2^{nd} ed.), Blackwell Scientific Publications (Philadelphia, PA), p. 722 (1976), incorporated by reference in their entireties. In examples, the cells are embedded, e.g., distributed throughout the bioactive scaffold, or enclosed within the bioactive scaffold (e.g., distributed evenly throughout the bioactive scaffold). The term “embedded”, as used herein, may generally refer to the placement and capture of cells within the bioactive scaffold.

In embodiments, the volume of the gel is distributed to an injury site, and is determined by the size and nature of the injury (regardless of whether the patient in question is a child or adult). The volume of the gel is sufficient to fill the defect (if applicable) or sufficient to interface with the surface area of a tissue tear (if applicable). In other examples, the number of cells used is determined by the volume of the gel administered. For example, the cells encapsulated in the gel is in the range of 1.0×10⁴ to 1.0×10⁷ per 10 μL of gel, and a preferred concentration of cells being 4.0×10⁶ per 10 μL of the gel. In embodiments, the number of cells is from about 1.0×10³ to 1.0×10⁶ cells/μL of gel, or from about 1.0×10⁴ to 5.0×10⁵ cells/μL of gel, or about 4.0×10⁵ cells/μL of gel. As used herein the number of cells is determined after the combining component 1 and component 2, for example the number of cells per μL is determined after the combination of component 1 and component 2.

In some embodiments, the thrombin in the bioactive scaffold (or compositions thereof) is in a concentration from about 10-1000 units/mL, or from about 350-700 units/mL, or from about 400-500 units/mL. In embodiments, the fibrinogen in the bioactive scaffold (or compositions thereof) is in a concentration from about 10-1000 mg/mL, or from about 60-120 mg/mL, or from about 100-110 mg/mL. In embodiments, the aprotinin in the bioactive scaffold (or compositions thereof) is in a concentration from about 1000-5000 KIU/mL, or from about 2250-3750 KIU/mL, or from about 2500-3500 KIU/mL. In embodiments, the calcium chloride in the bioactive scaffold (or in compositions thereof) is in a concentration from about 5-100 μm/mL, or from about 10-60 μm/mL, or from about 30-50 μm/mL. In embodiments, the SDF-1 in the bioactive scaffold (or compositions thereof) is in a concentration from about 1-100 ng/mL, or from about 1-50 ng/mL, or about 30 ng/mL. In embodiments, the KGN is first dissolved in DMSO, and after it is dissolved in the DMSO, it is serially diluted in distilled water to reach the desired concentration, e.g., from about 0.01-0.9 μg/mL, or from about 0.01-0.5 μg/mL, or about 0.1 μg/mL.

In aspects, provided herein is a composition for repairing a musculoskeletal tissue defect or injury comprising a population of cartilage-derived mesenchymal progenitor cells (CPCs), wherein the CPCs express cell surface markers comprising CD166, CD54, or CD105, and wherein the CPCs do not express the cell surface markers comprising CD106, CD4, CD14, or CD34.

In aspects, provided herein is a bioactive scaffold composition comprising a population of cartilage-derived mesenchymal progenitor cells (CPCs) (e.g., the population of cells comprises a suspension of cells), fibrinogen, thrombin, stromal cell derived factor 1 (SDF-1), and kartogenin (KGN). In some examples, the cells (e.g., the CPCs) are at least 50%, 60%, 75%, 80%, 90%, 95%, 98%, or 99% purified. In some examples, the population of cells, e.g., a purified population of cells do not comprise bone-marrow derived stem cells, and in other examples, the purified population of CPCs contain less than 10%, less than 1%, less than 0.1%, or less than 0.01% of bone-marrow derived stem cells.

In other embodiments, the CPCs (e.g., a population of CPCs or a purified population of CPCs or purified CPCs) express SOX9 relative to bone marrow-derived stromal cells (BMSCs), or wherein the CPCS express less COL10 relative to BMSCs.

In an embodiment of the invention, the cell population (e.g., the isolated or purified CPC cell population) comprises a mixture of cells expressing any of the biomarkers described herein. For example, the isolated or purified cell population express cell surface markers comprising CD166, CD54, or CD105, and wherein the CPCs do not express the cell surface markers comprising CD106, CD4, CD14, or CD34. In other embodiments, the cell population (e.g., the isolated or purified CPC cell population) expresses greater SOX9 relative to bone marrow-derived stromal cells (BMSCs), or wherein the CPCs express less COL10 relative to BMSCs. For example, the cell population expresses greater than 10%, 1%, 0.1%, or 0.01% increase in SOX9 mRNA compared to BMSCs, where SOX9 expression held as 1.0 (for comparison). In other examples, the CPCs express greater than a 1.0 fold, 1.2 fold, 1.5 fold, 2 fold or more increase in COL10 mRNA compared to BMSCs, where COL10 expression held as 1.0 (for comparison). For example, the cell population expresses less than 10%, 1%, 0.1%, or 0.01% increase in COL10 mRNA compared to BMSCs, where COL10 expression held as 1.0 (for comparison). In other examples, the cell population expresses less than a 1.0 fold decrease in COL10 mRNA, COL10 expression held as 1.0 (for comparison).

The population of cells (cartilage-derived mesenchymal progenitor cells (CPCs) are processed or purified. Specifically, as used herein, an “isolated” or “purified” refers to a cell population that is substantially free of other cell types or cellular material. Purified also defines a degree of sterility that is safe for administration to a human subject. The purified cells demonstrate a high degree of viability, both before and after storage at 4° C. or under liquid nitrogen, and after being shipped at temperatures less than 12° C. or on ice. In certain embodiments, the percentage of viable cells, as determined by standard methods (e.g., by an MTT assay), is at least 50%, 60%, 70%, 80%, 90%, 95%, 99%, or 100%. In related embodiments, the percentage of viable cells, as determined by standard methods (e.g., by an MTT assay), following storage at 4° C. for 24 hours or storage under liquid nitrogen for two weeks is at least 40%, 50%, 60%, 70%, 80% or 90%. In another embodiment, the percentage of viable cells, as determined by standard methods (e.g., by an MTT assay), following refrigerated storage or shipment on ice packs for less than 24 hours is at least 30%, 40%, 50%, 60%, 70%, 80%, 90%, 95%, 99%, or 100%.

The term “isolated” as used herein means having been removed from its natural environment. For example, the population of cells are isolated from an anatomical location or developmental lineage, e.g., cartilage, and comprise less than 10%, less than 1%, less than 0.1%, or less than 0.01% of cells from a different anatomical location or developmental linage. For example, the isolated CPCs or CPC cell line comprise less than 10%, less than 1%, less than 0.1%, or less than 0.01% of bone-marrow derived cells. In other examples, the population of cells (e.g., the CPC population) contains less than 10%, less than 1%, less than 0.1%, or less than 0.01% of mature fully differentiated cartilage cells. The term “purified” as used herein means having been increased in purity, wherein “purity” is a relative term, and not to be necessarily construed as absolute purity. For example, the purity can be at least about 50%, can be greater than 60%, 70% or 80%, 90% or can be 100%.

The population of cells described herein (e.g., the CPCs) can also be substantially purified. The term “substantially purified” as used herein refers to a population of cells (e.g. CPCs) that are substantially enriched in a sample. The sample can be substantially purified or enriched for the CPCs of interest such that the sample is at least about 50%, 60%, 70%, 80%, 85%, 90%, 95%, 99% or greater of the desired CPCs or less than about 40%, 30%, 20%, 15%, 14%, 13%, 12%, 11%, 10%, 9%, 8%, 7%, 6%, 5%, 4%, 3%, 2%, 1% or less of the undesirable or other cells present.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about. About with respect to concentration range of the compositions and methods of the current disclosure also refers to any variation of a stated amount or range which would be an effective amount or range.

The compounds or components of the therapeutic mixtures (e.g., fibrin glue with SDF-1, KGN and CPCs are processed or purified. For example, polynucleotides, polypeptides, or other agents are purified and/or isolated. Specifically, as used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. Purified also defines a degree of sterility that is safe for administration to a human subject, e.g., lacking infectious or toxic agents.

Similarly, as used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) or polypeptide is free of the amino acid sequences or nucleic acid sequences that flank it in its naturally-occurring state. Purified also defines a degree of sterility that is safe for administration to a human subject, e.g., lacking infectious or toxic agents.

A small molecule is a compound that is less than 2000 Daltons in mass. The molecular mass of the small molecule is preferably less than 1000 Daltons, more preferably less than 600 Daltons, e.g., the compound is less than 500 Daltons, 400 Daltons, 300 Daltons, 200 Daltons, or 100 Daltons.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The data described herein demonstrate that CPCs, SDF-1, and KGN encapsulated in a fibrin glue scaffold can be used to fill a tissue defect. The scaffold described above was used to fill a meniscus tissue defect (cartilaginous tissue). This approach takes advantage of the regenerative effects of the exogenously administered CPCs (placed inside the scaffold), and also the regenerative effects of endogenous native progenitor cells that migrate to the defect site from the hosts own body, in response to the chemoattractant SDF-1. The SDF-1 also acts as a mitotic agent that enhances cell viability with the fibrin scaffold (FIG. 9). This (the increased viability of the cells) was an unexpected and surprising effect of SDF-1 and fibrin glue scaffold that was discovered. The KGN acts as a chondrogenic factor that initiates chondrogenesis in both exogenous CPCs and endogenous cells to induce the formation of cartilaginous neo-tissue that fills the defect site.

The invention represents an improvement over the current paradigm of using bone-marrow derived stem cells (BM-MSCs) alone in conjunction with KGN to stimulate cartilage repair, because the data shows that CPCs are able to undergo chondrogenesis to a greater degree than BM-MSCs (as shown in FIGS. 3 and 5). Indeed, CPCs are the most chondrogenic of all mesenchymal stem cells (MSCs). These findings indicate that the neo-tissue formed by CPCs in response to KGN are more cartilaginous (containing more Collagen 2, which is essential for the compressive properties of cartilage and fibrocartilage tissues). Other features and advantages of the invention will be apparent from the following description of the preferred embodiments thereof, and from the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### DESCRIPTION OF DRAWINGS

FIG. 1A is schematic diagram of tissue repair method using Fibrin Glue, SDF-1 and CPCs.

FIG. 1B is a schematic diagram of a method of preparation for Fibrinogen and Thrombin injectable product for therapeutic use.

FIG. 2 is schematic diagram of KGN cell treatment and evaluation cell response to treatment.

FIGS. 3A and dB are bar graphs showing relative mRNA expression levels to untreated MSCs for (FIG. 3A) markers of chondro/fibrochondro-genesis and (FIG. 3B) chondrocyte hypertrophy. *p<0.05 **p<0.01 **p<0.001 ****p<0.0001.

FIG. 4 is a pair of bar graphs showing protein concentration in CPC vs. MSC cell culture supernatant following treatment with KGN. *p<0.05 **p<0.01 **p<0.001 ****p<0.0001 2-way ANOVA with Dunnett's multiple comparisons test.

FIG. 5 is a bar graph showing results of Alcian blue staining of CPC vs. MSC following 10d treatment with KGN. *p<0.05 **p<0.01 **p<0.001 ****p<0.0001 2-way ANOVA with Dunnett's multiple comparisons test.

FIG. 6A is a schematic diagram of an ex vivo model of a mammalian meniscus tissue defect.

FIG. 6B is a photograph of an apparatus used for a force displacement test of 2 mm indenter through fibrin glue construct within meniscus explant.

FIG. 7 is a bar graph showing results of Push-Through Testing.

FIG. 8 is an image showing that cartilage progenitor cells (CPCs) are a subpopulation of skeletal tissue derived mesenchymal progenitor cells (STMSCs) native to articular cartilage tissue. The have higher basal chondrogenic potential than marrow-derived stem cells (BM-MSCs), hence they are ideal for cartilaginous tissue repair applications.

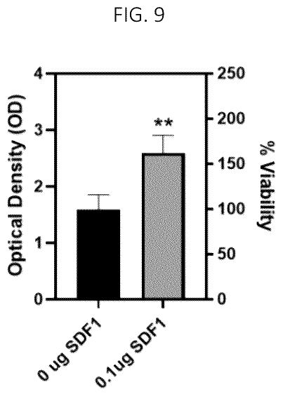

FIG. 9 is a bar graph showing that SDF-1 infused hydrogel increased CPC viability. A quantitative measurement of viable cells using an MTT (3-(4,5-dimethylthiazol-2-yl)-2,5-diphenyltetrazolium bromide) assay after two weeks of encapsulation in fibrin hydrogel sealant containing rSDF-1 (μg/mL), relative to control group without rSDF-1. N≥4. **P≤0.01. The cell viability was increased by up to 40% in the presence of 0.1 μg of SDF-1 in a 20 μl fibrin gel. Cells were seeded at a density of 25,000 cells per fibrin gel construct.

FIG. 10A and 10B are data showing that CPC treatment stimulated filling of meniscus tears in a live rodent model. FIG. 10A are images showing cell-mediated healing of isolated medial meniscus tears in rats 46 days following treatment with CPCs, BM-MSCs, or no cells. 3.2 million fluorescently labeled cells were injected into the joint capsule following meniscus injury. Cells from the treatment are fluorescently labeled (red). Nuclei of all cells (both native meniscus cells and injected cells) are labeled with Dapi (blue). Yellow lines and arrows signify areas that remain open without healing, with yellow dotted lines inscribing the unfilled areas. Scale bar=100 μm. FIG. 10B is a bar graph showing the percent tear filling (by area) resulting from treatment with CPCs or BM-MSCs. The no-cell control group exhibited 0% filling. N≥3. ###, P≤0.005 compared to control group. **, P≤0.01.

FIGS. 11A-11C are data showing that CPC treatment of meniscus tears reduced osteoarthritis (OA) changes in the knee. FIG. 11A are images showing knee articular cartilage of rats that have had a meniscal tear treated with CPCs, BM-MSCs, or vehicle alone (saline control). Sections were stained with Safranin O/Fast green, hematoxylin. Scale bar=100 μm. The saline-treated control animals and BM-MSC-treated animals exhibited noticeably more cartilage degeneration and proteoglycan loss. FIG. 11B is a bar graph showing the Osteoarthritis Research Society International (OARSI) histopathology scoring of the medial femoral condyle. FIG. 11C is a bar graph showing that the medial tibial plateau demonstrated a statistically significant difference between BM-MSC-treated and CPC-treated animals. A nonparametric ANOVA was used to perform statistical analysis. N≥3. *, P≤0.05; **, P≤0.01; ***, P≤0.005.

FIG. 12 are images showing that SDF-1 pre-treated scaffolds can be used to localize CPCs to sites of tissue injury. rSDF-1 pretreated (left panels) and untreated (right panels) HPC scaffolds seeded with red fluorescently labeled CPCs (1.0×10⁵) were secured between two human meniscus tissue plugs (4 mm diameter) to represent a scaffold placed at the site of a meniscus tissue break/tear. Constructs were incubated in culture. Red channel and brightfield channel were acquired after two weeks in culture and merged. “M” signifies meniscus plugs and “S” signifies scaffold. Images demonstrate the increased retention of CPCs in scaffolds containing rSDF-1.

FIG. 13 are images showing that a fibrin hydrogel containing CPCs and SDF-1 stimulated healing of meniscal tears in Yucatan Minipigs. Representative macroscopic images of meniscus tears treated under three experimental conditions, three months following injury. Images are shown in the sagittal plain of the medial menisci. The left panel shows the tear repaired with suture alone; the middle panel shows the tear repaired with suture followed by treatment with fibrin hydrogel that does not contain CPCs; the right panel shows the tear repaired with suture followed by treatment with fibrin hydrogel that contains CPCs.

### DETAILED DESCRIPTION

The invention is a multicomponent bioactive scaffold that stimulates the proliferation, differentiation, and maturation of exogenously administered stem cells (as well as native migrating stem cells) in order to facilitate accelerated healing of musculoskeletal soft-tissue defects (i.e. meniscus, tendon, ligament, and craniofacial tissues).

The compositions and methods described herein provide a solution to long standing problems regarding repair of musculoskeletal joint injuries or disorders. Fibrocartilage tissue defects in joints such as the knee and temporomandibular joint (TMJ) cause post-traumatic arthritis that lead to chronic joint degeneration. This invention accelerates the formation of fibrocartilaginous neo-tis sue at the site of large fibrocartilage tissue defects that otherwise will not heal in time before the onset of arthritis.

The method of creating a bioactive scaffold entails combining fibrinogen and thrombin to encapsulate a homogenous solution of skeletal tissue derived mesenchymal progenitor cells (STMSCs), stromal cell derived factor 1 (SDF-1), and kartogenin (KGN) to form a chondrogenic fibrin glue delivered to individuals at the site or injury or degeneration. The fibrin glue scaffold is injected or arthroscopically placed at the injury site one time, followed by additional injections of STMSCs into the joint bi-weekly or monthly (if necessary). This bioactive scaffold is designed to be an injectable and it can be accordingly delivered and localized directly to musculoskeletal tissue injury sites.

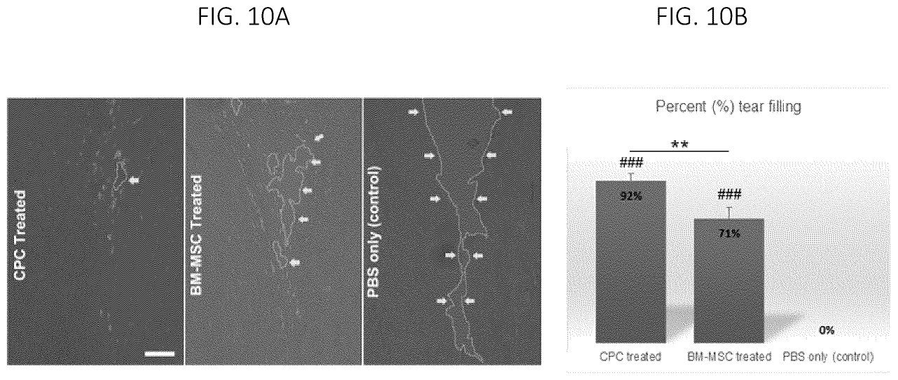

In other embodiments, the SDF-1 provides for a synergistic effect of SDF-1 and fibrin glue scaffold. In embodiments, the term “synergistic” refers an increase in the regenerative effects, after the use of the bioactive scaffold in combination with the SDF-1, which is significantly higher than the regenerative effects of the bioactive scaffold when used without SDF-1. In other words, the addition of the SDF-1 to the bioactive scaffold provides for a synergistic effect (not merely additive) for regenerative purposes, as compared to the bioactive scaffold without the presence of SDF-1. For example, the regenerative effect of including SDF-1 in the bioactive scaffold is at least 1.5×, 2×, 3×, 4× or higher as compared to the regenerative effect of the bioactive scaffold without the SDF-1. In other examples, the SDF-1 provided the chemotactic effect wherein the SDF-1 influenced cells to migrate to the defect site (where chemotaxis (or chemotactic effect) generally refers to the number of cells found to have migrated to a certain location). An unexpected and surprising result of the SDF-1, however, was that in addition to having the chemotactic effect, the cells also showed an increased viability (e.g., as determined by an MTT assay). For example, the cells showed greater than a 50% increase in cell viability relative to the untreated hydrogel control group (e.g., without SDF-1, see FIG. 9). In other examples, the cells show great than a 60%, 70%, 80%, 90% increase in cell viability relative to the untreated hydrogel control group (without SDF-1).

The methods and compositions as associated with advantages compared to prior or existing approaches to musculoskeletal joint repair. For example, the invention provides an injectable carrier of therapeutic progenitor/stem cells with an optimized combination of chemotactic and chondrogenic factors to: 1) facilitate retention of the exogenously administered STMSCs; 2) induce migration of native progenitor cells from the patient's own body to the site of injury; and 3) stimulate proliferation and differentiation of these cells into neo-tissue that will fill and repair the tissue defect. Overall, this invention promotes tissue healing. Intra-articular injection of cells to the joint space have been previously implemented in pre-clinical and clinical settings. However, without an appropriate carrier, cells experience massive dispersion from the site of injury, sharply decreasing efficacy. In the design described here, the fibrin glue adheres to the site of injury, SDF-1 chemo-attracts native and exogenous stem cells to the site of introduction, and kartogenin enhances proliferation and differentiation of stem cells to fibrocartilaginous phenotypes.

The invention promotes the progressive filling of tissue defects with fibrocartilaginous neo-tissue allowing for accelerated healing of these injuries. Specifically, this invention is used to accelerate fibrocartilage tissue repair and restoration that is highly relevant for healing meniscus tissue injuries in the knee joint and TMJ fibrocartilage. This compositions and methods are also useful to treat articular cartilage defects, themselves.

The compositions and methods described herein promote the progressive filling of tissue defects with fibrocartilaginous neo-tissue through a system of delivering and localizing exogenous stem cell progenitors to the site of injury.

### Assembly of Injectable Stem-Cell Construct

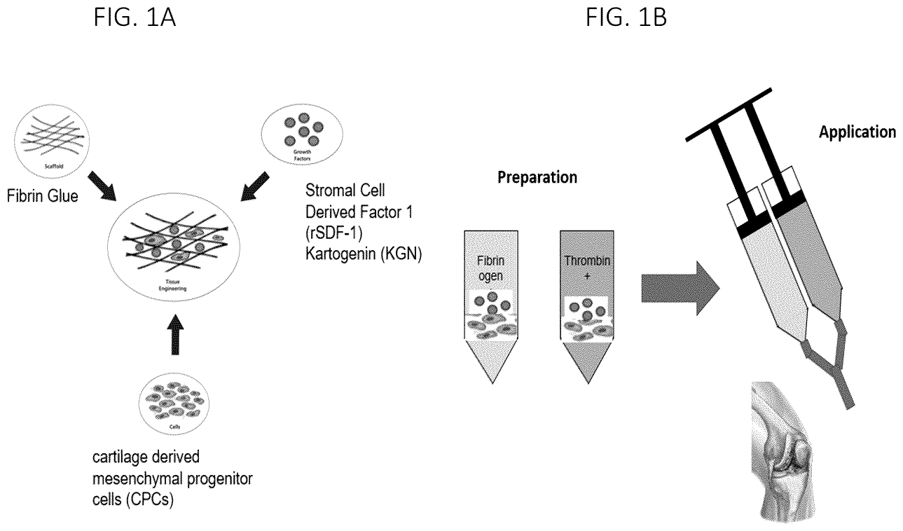

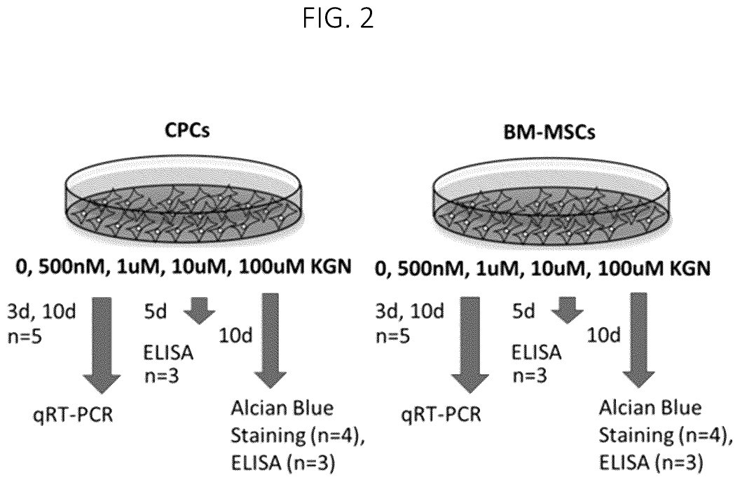

The injectable stem cell composition involves three components: 1. cells (e.g., cartilage derived mesenchymal progenitor cells (CPCs), 2. growth and chemotactic factors (Kartogenin (KGN)) and recombinant stromal cell derived factor 1 (rSDF-1) respectively), and 3. Fibrin glue (FIG. 1A).

The CPCs are as described below. Methods of making CPCs are described in U.S. Pat. No. 10,130,687.

KGN is a RUNX1 transcriptional activator and binds filamin A. The structure of KGN is shown below:

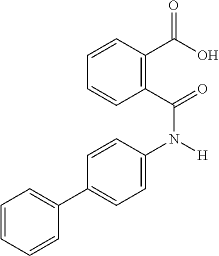 [SMILES: O=C(O)c1ccccc1C(=O)Nc1ccc(-c2ccccc2)cc1]

KGN is commercially available from Millipore Sigma.

The fibrin glue (Baxter Healthcare Corporation (Glendale, California)), which is constituted through the combination of a fibrinogen and thrombin.

When the fibrinogen and thrombin are homogenized, calcium and factor XIII within the solution aid in the conversion of fibrinogen into insoluble fibrin. Thus, these two separate components of fibrinogen and thrombin are each homogenized separately with, e.g. most effective. 0.1 μg of SDF-1, has the greatest chemotactic effect in what has been tested.

For example, clinical dosage range from 10 ng/mL-1 μg/mL to cover a broader range of doses. The justification could be that depending on the number of cells used in the fibrin hydrogel, the range of SDF-1 can be increased or decreased accordingly. 30 ng/mL to 0.1 μg/mL rSDF-1, 1 μM KGN (Sigma-Aldrich, Burlington, MA), and 1×10⁶ cells/mL of CPCs, an enriched cell line of which was have developed (Accession Number: PTA-127250) from the full thickness of human articular cartilage. Each respective component is loaded into separate chambers of a dual injector, and when depressed each component mixes at the size of tissue defect (FIG. 1B).

### ATCC Deposit

In embodiments, the cartilage progenitor cell line used herein is the Cartilage Progenitor Cell Line 2 (CPCL2) that has been deposited under the terms of the Budapest Treaty with American Type Culture Collection (ATCC) and 37 C.F.R. § 1.803(a)(1), where the name and address of the International Authority is: ATCC located at 10801 University Boulevard in Manassas, Virginia 20110-2209. The deposited cell line (CDCL2) has been given the following Patent Deposit Number (Accession Number): PTA-127250, and was deposited on Jan. 27, 2022. The cell line will be irrevocably and without restriction of condition released to the public upon issuance of a patent and that the cell line will be replaced if the deposited cells ever becomes non-viable. A sub-colony of the stable human cartilage progenitor cell line (HCPCL) was used to conduct all experiments including CPCs.

### Assessing Function of KGN-rSDF1-CPCs Fibrin Glue Construct

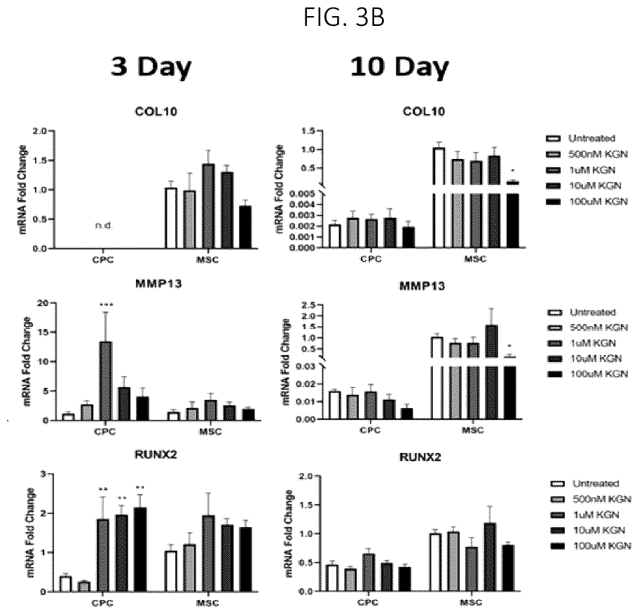

1 μM KGN enhances chondrogenesis of CPCs but not MSCs. The following materials and methods were used to generate the data described herein. Human CPCs and bone marrow derived mesenchymal stem cells (BM-MSCs) were cultured at 40,000 cells/well for 3 days or 22,000 cells/well for 10 days on 12-well plates. Cells were either untreated or treated with 500 nM, 1 μM, 10 μM, or 100 μM KGN (Sigma-Aldrich) at N=5 for each treatment group. At experimental endpoint following KGN treatment, RT-qPCR was used the measure expression of chondrogenic markers SRY-Box Transcription Factor 9 (SOX9), collagen 2 (COL2), collagen 1 (COL1) (FIG. 3A) as well as hypertrophy and catabolism markers, associated with development of osteoarthritis, collagen 10 (COL10), matrix metallopeptidase 13 (MMP13), Runt-related transcription factor 2 (RUNX2) (FIG. 3B).

CPCs and BM-MSCs were cultured again at 22,000 cell/swell for 10 days in 12-well plates and again treated with KGN. At 5 and 10 days of treatment, cell culture supernatant was collected, with cell media and KGN replenished at the 5-day timepoint (N=3). Supernatants were used to perform ELISA for measuring MMP13 protein. At day 10, cell culture plates were stained for Alcian Blue (N=4). The color was extracted and the absorbances were quantified via spectrophotometry, to assess acidic glycosaminoglycans (GAG) and hyaluronan (HA). In analysis, within and between group differences were assessed through 2-way ANOVA with Dunnett's multiple comparisons test. Significant differences are signified by p-values ≤0.05.

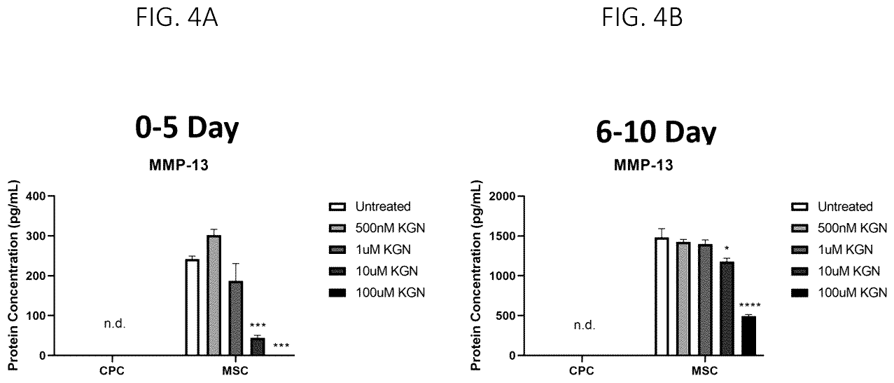

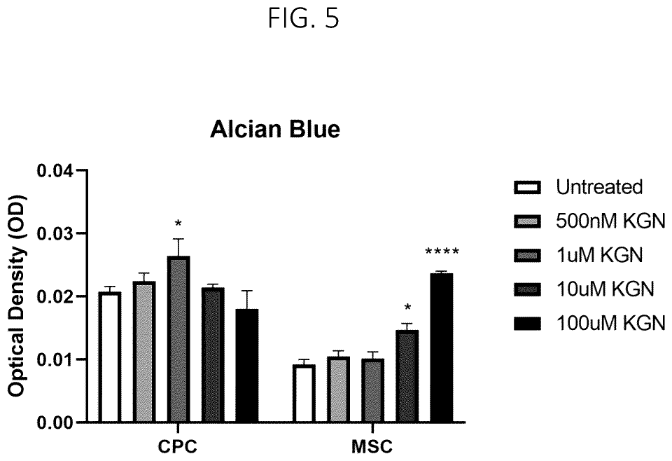

Treatment of CPCs with 1 μM KGN led to 2-fold upregulation of SOX9 (p<0.05) and COL2 (p<0.0001) at 3 days (FIG. 3A). This upregulation of COL2 in response to 1 μM of KGN was maintained at 10 days of treatment (p<0.05). KGN did not increase expression of SOX9, COL2, or COL1 in BM-MSCs. Regarding markers of chondrocyte hypertrophy, at baseline CPC had no detectable expression of COL10 at 3 days, and at 10 days expression of COL10, MMP13, and RUNX2 were significantly less than that of BM-MSCs for all treatment groups (p<0.05) (FIG. 3B). For BM-MSCs, 100 μM KGN significantly reduced expression of COL10 and MMP13 at 10 days (p<0.05). At 3 days, 1 μM KGN increased expression of MMP13, and 1 μM, 10 μM, and 100 μM KGN increased expression of RUNX2. However, both between 0-5 days KGN treatment and 6-10 days KGN treatment, supernatant collected from CPCs at all treatment groups contained undetectable levels of MMP13 protein (FIG. 4). Conversely, parallel to mRNA expression results, 10 μM and 100 μM KGN led to significant decrease in protein levels of MMP13 both at 0-5 days (10 μM: p<0.001, 100 μM: p<0.0001) and 5-10 days (10 uM: p<0.05, 100 μM: p<0.0001) following KGN treatment. Alcian Blue stained CPCs treated with 1 μM KGN with increased intensity at 10 days compared to untreated CPCs (p<0.05) (FIG. 5). BM-MSCs treated with 10 μM (p<0.05) and 100 μM KGN (p<0.0001) stained more intensely than BM-MSCs that were untreated.

The following conclusions were drawn from these studies.

The data described herein indicated that KGN was a poor chondrogenic factor for human BMSCs. KGN's only effect has shown to be downregulation of markers for chondrocyte hypertrophy, catabolism, and osteoarthritis development. Instead, KGN, especially at 1 μM concentration, can further enhance chondrogenic ability of human CPCs (see, Zhou Q, et al. Med Sci Monit. 2019 Jul. 4; 25: 4960-4967, and Music E et al. Sci Rep. 2020 May 20; 10(1):8340, incorporated herein by reference in their entireties.

### KGN Increases Mechanical Integrity of a rSDF1-CPC Fibrin Glue Construct Ex Vivo

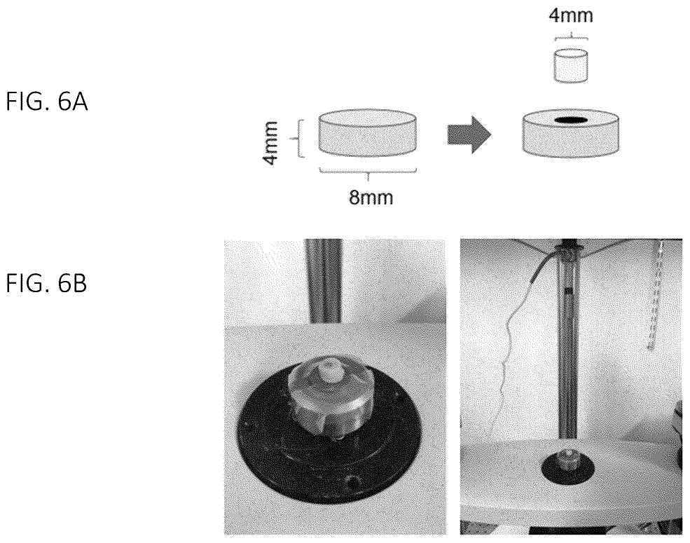

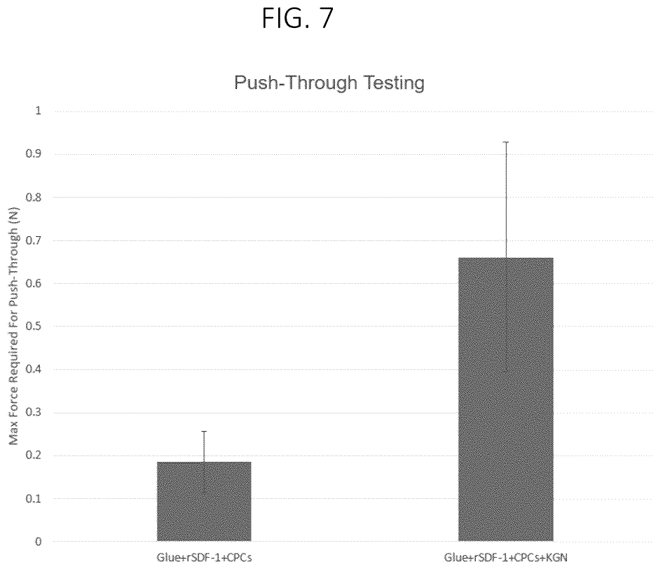

Cylindrical 8 mm diameter tissue cores were punched out of bovine lateral and medial menisci (herein referred as Punches) and cut to uniform height of 5 mm. From these punches, a 3 mm diameter inner core was again removed to simulate a full thickness meniscal defect (FIG. 6A). Fibrinogen Thrombin with rSDF-1 (30 ng/mL), KGN (1 uM), and CPCs (1e6/mL) were added to fibrinogen to form the bioactive scaffold, which was used to fill the 3 mm defect. Equal quantities of thrombin solution and fibrinogen solution were used (12.5 μl each), forming a homogeneous fibrin glue (n=6). This was compared to a preparation of the fibrin glue without KGN (n=5). The explants were placed in a 37° C. incubator with DMEM with 10% FBS, 1% Pen Strep, 100 mM HEPES, 2 mM L-glutamine, 0.1 mM ascorbic acid, 0.1 mM sodium pyruvate, 2.7 μM L-glucose (DMEM++) cell culture medium.

After 21 days, the explant punches were tested for integration of the full-thickness defect using a loading frame (ELF 3200, EnduraTec, Minnetonka, MN) (FIG. 6B). A 2 mm piston connected to the frame and centered above the fibrin glue inner core. The frame lowered the piston to a final displacement of 4 mm at a rate of −0.083 mm/sec. MATLAB (Mathworks, Natick, MA) is used to create force-displacement curves, with max force of “failure” of construct defined as maximum force registered in pushing through the construct.

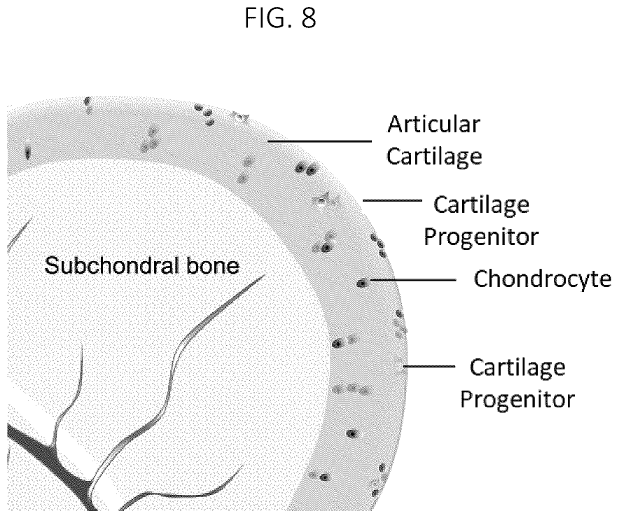

The constructs with fibrin glue, rSDF-1, CPCs, and KGN required a max force of 0.6610 N of force to push-through with standard error of the mean (SEM) of 0.267 N (FIG. 7). The constructs with fibrin glue, rSDF-1, CPCs required a max force of 0.1845 N to push-through with standard error of the mean (SEM) of 0.0714 N. The data herein show the trend of increasing force.

Explants are processed for histological evidence of neo-tis sue and fibrocartilage. Further, the biomechanical assay is used to test explants at 14 days of incubation. Data at 21 days of incubation indicates that the addition of KGN to a fibrin glue, rSDF-1, and CPC scaffold enhances mechanical integrity of the center construct of the explant. This is relevant because it implied that the fibrin glue scaffold had superior integration with the surrounding meniscus tissue when KGN was included. From a clinical standpoint, these findings insinuate that treating a meniscal defect in this manner would lead to stronger integration and better defect repair. Such outcomes are confirmed in the clinic using magnetic resonance imaging (MRI), computed tomography (CT) scanning, and histology analysis of the meniscus and underlying joint cartilage.

### EXAMPLE 1

### Kartogenin Induced Chondrogenesis in Cartilage Progenitor Cells and Attenuated Cell Hypertrophy in Marrow-Derived Stromal Cells for Application in Meniscus Tissue Repair

Meniscal injuries increase risk of osteoarthritis (OA), a chronic degenerative disease characterized by chondrocyte hypertrophy and tissue catabolism. Low vascularity of the meniscal fibrocartilaginous tissue lends to poor healing. Cell-based therapies to aid in meniscus repair have shown promise. However, successfully driving chondrogenesis of stem/progenitor cells upon administering them to the site of injury, while simultaneously avoiding phenotypic changes that favor a catabolic microenvironment, remains an important goal for cell-based cartilage and fibrocartilage repair strategies. While transforming growth factor β (TGFβ) has been a standard method to induce chondrogenesis though at the cost of chondrocyte hypertrophy at later time points, which is a phenotype that precedes cellular senescence and apoptosis, a newer non-biologic agent, kartogenin (KGN) may be a viable alternative that does not have the same regulatory hurdles. KGN has been shown to induce chondrogenesis at lower concentrations in marrow-derived stromal cells (BM-MSCs) as well as protect against inflammation through upregulation of lubricin expression. Currently, KGN's effect on cartilage-derived progenitors (CPCs), which have high potential for stimulating meniscal fibrocartilage healing, is unknown. Similarly, KGN's effects on chondrocyte hypertrophy have not yet been studied. It was hypothesized that CPCs would undergo chondrogenesis at an early timepoint compared to BM-MSCs in response to KGN and that KGN would decrease markers of chondrocyte hypertrophy in both cell types.

### Methods

Human CPCs and BM-MSCs were cultured at 40,000 cells/well for 3 days or 22,000 cells/well for 10 days on 12-well plates. Cells were either untreated or treated with 500 nM, 1 uM, 10 uM, or 100 uM KGN (Sigma-Aldrich) at N=5 for each treatment group. At experimental endpoint following KGN treatment, RT-qPCR was used the measure expression of chondrogenic markers SOX9, COL2, COL1 as well as hypertrophy and catabolism markers COL10, MMP13, RUNX2. CPCs and BM-MSCs were cultured again at 22,000 cell/swell for 10 days in 12-well plates and again treated with KGN. At 5 and 10 days of treatment, cell culture supernatant was collected, with cell media and KGN replenished at the 5-day timepoint (N=3). Supernatants were used to perform ELISA for measuring MMP13 protein. At day 10, cell culture plates were stained for Alcian Blue (N=4). The color was extracted and the absorbances were quantified via spectrophotometry, to assess acidic glycosaminoglycans (GAG) and hyaluronan (HA). In analysis, within and between group differences were assessed through 2-way ANOVA with Dunnett's multiple comparisons test. Significant differences are signified by p-values≤0.05.

### Results

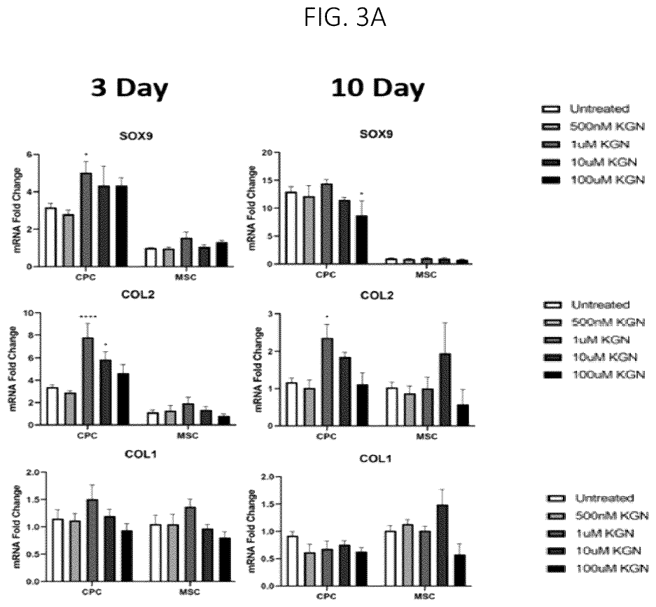

Treatment of CPCs with 1 μM KGN led to 2-fold upregulation of SOX9 (p<0.05) and COL2 (p<0.0001) at 3 days (FIG. 1A). This upregulation of COL2 in response to 1 uM of KGN was maintained at 10 days of treatment (p<0.05). KGN did not increase expression of SOX9, COL2, or COL1 in BM-MSCs. Regarding markers of chondrocyte hypertrophy, at baseline CPC had no detectable expression of COL10 at 3 days, and at 10 days expression of COL10, MMP13, and RUNX2 were significantly less than that of BM-MSCs for all treatment groups (p<0.05) (FIG. 1B). 100 μM KGN significantly reduced expression of COL10 and MMP13 at 10 days (p<0.05) For BM-MSCs. At 3 days, 1 μM KGN increased expression of MMP13, and 1 uM, 10 uM, and 100 uM KGN increased expression of RUNX2. However, both between 0-5 days KGN treatment and 6-10 days KGN treatment, supernatant collected from CPCs at all treatment groups contained undetectable levels of MMP13 protein (FIG. 2). Conversely, parallel to mRNA expression results, 10 μM and 100 uM KGN led to significant decrease in protein levels of MMP13 both at 0-5 days (10 μM: p<0.001, 100 μM: p<0.0001) and 5-10 days (10 μM: p<0.05, 100 μM: p<0.0001) following KGN treatment. Alcian Blue stained CPCs treated with 1 μM KGN with increased intensity at 10 days compared to untreated CPCs (p<0.05). BM-MSCs treated with 10 μM (p<0.05) and 100 μM KGN (p<0.0001) stained more intensely than BM-MSCs that were untreated.

### Discussion

The present study found that KGN enhanced chondrogenesis in CPCs, as seen by increased expression of chondrogenesis markers. For BM-MSCs, 100 μM KGN led to decreased expression and production of MMP13 as well as hypertrophic marker COL10. Interestingly, KGN had no chondrogenic effect in BM-MSCs, an observation that adds to growing evidence that KGN may have few effects on human BM-MSCs (Music et al. Sci Rep. (2020) 10:8340). Prior work has primarily investigated KGN's effect on porcine cells, highlighting species differences may be a relevant factor. This study also showed that CPCs may be a viable cell-based therapy for use in conjunction with KGN for tissue repair. Not only were CPCs found to have higher expression of chondrogenic markers and lower expression of hypertrophy markers at baseline, but also 1 uM KGN treatment results in enhanced chondrogenesis, as seen with increased Alcian Blue staining at 10 days. Of note, while 1 μuM KGN treatment increased expression of MMP13, there was undetectable MMP13 protein production. This is consistent with MMP13's role here, not as an extracellular metalloproteinase, but as a marker of chondrocyte maturation and tissue homeostasis in early chondrogenesis (see, Borzi et al. Arthritis Rheum (2010) 62(8): 2370-2381).

The results of this study suggest that CPCs that have been exposed or are exposed to KGN (in the range of 0.01-9.9 μM) may be used for cell-based therapies for meniscus repair. In other examples CPCs that have been or are exposed to KGN in an amount of 0.02 μM, 0.03 μM, 0.04 μM, 0.05 μM, 0.06 μM, 0.07 μM, 0.08 μM, 0.09 μM, 0.1 μM, 0.5 μM, 1 μM, 5 μM, 6 μM, 7 μM, 8 μM, 9 μM, or 10 μM.

It also suggests that treating endogenous CPCs, that exist in patients' body (i.e. inside the cartilage tissue), with KGN in the described dose range may be used to promote chondrogenic extracellular matrix synthesis. Further research is needed to study the efficacy of this combination at the tissue level, for example through ex-vivo and in-vivo models of fibrocartilage (i.e. meniscus) and cartilage repair in a clinically relevant large animal model such as porcine or ovine.

Human CPCs undergo increased chondrogenesis in response to KGN, whereas BM-MSCs do not, only undergoing attenuated cellular hypertrophy. Transplantation of CPCs with 1 uM KGN may be translatable to repair of human fibrocartilage tissues. In embodiments clinical advantages of the methods and compositions (e.g., comprising the CPCs described herein) include that the BM-MSCs do not respond to KGN, at least do not respond as much as the CPCs.

### EXAMPLE 2

### Efficacy of Cartilage-Derived Progenitor Cells (CPCs) for Repairing Meniscus Injuries

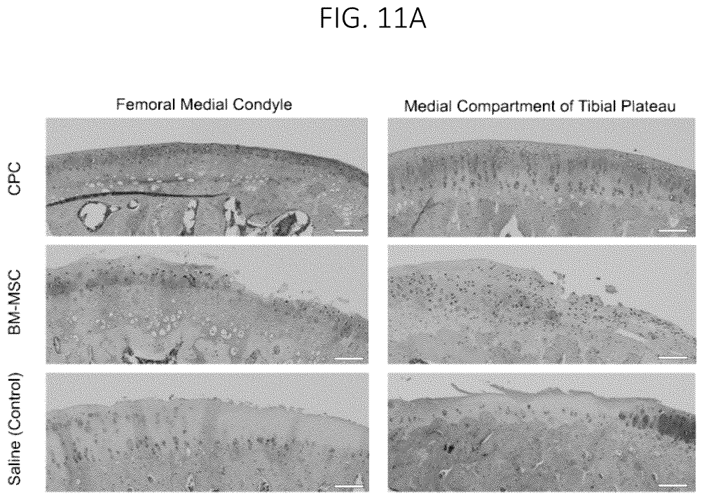

A rat medial meniscus injury model was used to evaluate CPC-stimulated meniscus healing in comparison to treatment with bone marrow stem cells (BM-MSCs) or treatment with saline/vehicle (control) only. A 1.5 mm longitudinal tear spanning the full thickness of the meniscus was created in the meniscus of the medial meniscus of 15-week-old skeletally mature rats. Fluorescently labeled human CPCs (1.6 million cells) were administered via intra-articular injection twice (7 and 28 days following initial surgery). There were two control groups: BM-MSC injected animal and vehicle (PBS) only injected animals. Rats were sacrificed and their medial menisci were evaluated using fluorescence imaging of the tear site 46 days post-surgery (FIG. 10A). Localization and engraftment of injected cells (labeled fluorescent red) to the tear site was confirmed on tissue sections. Analyses showed that CPC-treated animals exhibited increased tear filling compared to the group treated with BM-MSCs (FIG. 10B). PBS-treated controls were interpreted to have a mean filling of 0%. This control group was used to normalize measurements for the other two experimental groups. These experiments suggested that CPC-treatment stimulated fibrocartilage tear healing in a live animal model.

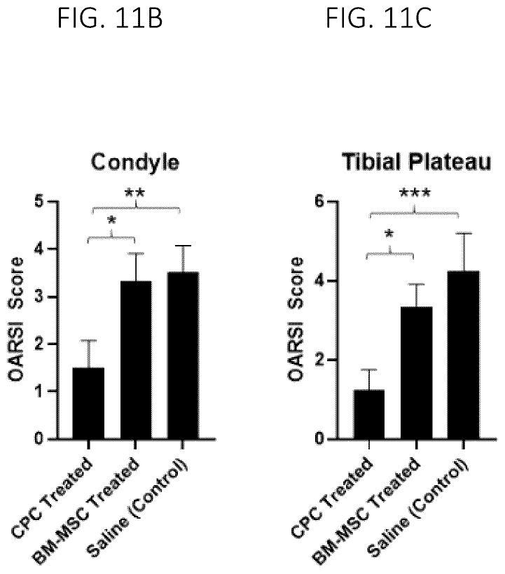

The articular surface of the medial knee compartment was analyzed as a secondary outcome measure of success in these animals. Animals treated with CPCs exhibited less cartilage damage in the medial compartment of the tibial plateau, which sits directly below the injured medial meniscus. The medial femoral condyles and medial tibial plateaus were sectioned and stained with Safranin-O/fast green (FIG. 11A). Osteoarthritis Research Society International (OARSI) histopathology scores demonstrated that the CPC-treated group exhibited a significant improvement over the vehicle only treated saline control group, whereas the BM-MSC-treated group did not (FIG. 11B and 11C).

Taken together, these data demonstrated that CPCs can be used to stimulate reintegration of damaged meniscal fibrocartilage tissue tears and protect articular joint cartilage from erosion. Thus, CPCs can be used as the cellular component of the hydrogel scaffold, which can also be used to stimulate soft tissue defect repair.

### EXAMPLE 3

### SDF-1 Increased CPC Viability and Facilitated Their Localization and Retention Within Targeted Sites of Interest

The in-vivo data described in the Examples 1 and 2 above suggested that injecting 3.2 million CPCs directly into the small knee joint spaces of rats was effective in stimulating a significant degree of meniscal healing. To further the understanding, a larger animal model was used. A drawback of using the larger animal model was that it has a significantly greater joint space where cells injected into the space can become dispersed and lost. Thus, it was necessary to find a means of delivering the cells in a targeted manner to the site of injury. To overcome this problem, it was found that recombinant SDF-1 (rSDF-1) can be used to direct CPCs to a particular region of interest.

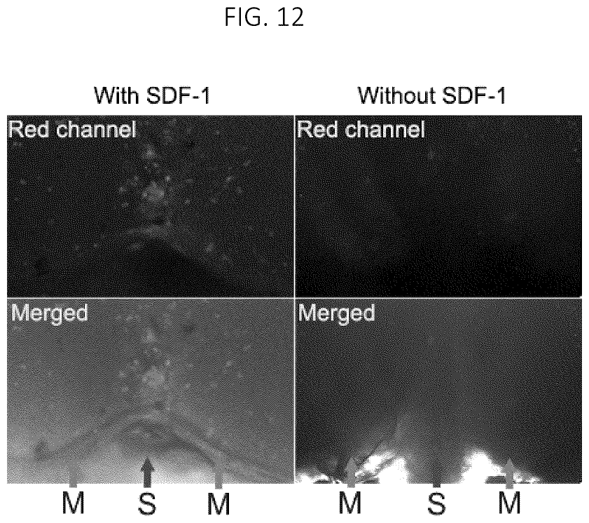

SDF-1 mediated cell migration is important for stimulating musculoskeletal tissue healing (see, e.g., Kawakami Y, et al. J Bone Miner Res. 2015; 30(1):95-105, Kitaori T, et al. Arthritis Rheum. 2009; 60(3):813-823, and Shen W, et al. Stem Cells Transl Med. 2014; 3(3):387-394, each of which is incorporated by reference in its entirety). Previously, it was demonstrated that the SDF-1 pathway activation is essential for mobilizing CPCs to injury sites in the rat meniscus, which is a crucial step in the CPC-mediated healing process (Jayasuriya C T, et al. Stem Cells. 2019; 37(1):102-114). In a recently published follow-up study, recombinant SDF-1 (rSDF-1) was successfully utilized to facilitate the localization of CPCs into a hydroxypropyl cellulose (HPC) scaffold, which was used for repairing damaged human meniscal tissue in explant culture (FIG. 12, reproduced from Newberry J et al. Connect Tissue Res. 2019; 61(3-4):338-348). This study established that CPCs are indeed SDF-1 responsive and that they can be rallied to spatial regions of interest using rSDF-1 to stimulate CPC chemotaxis.

Additionally, data also strongly suggested that rSDF-1 treatment of CPCs significantly increased their cell viability and/or proliferation, as determined by a tetrazolium (MTT) assay (FIG. 9). CPCs that were seeded into a hydrogel consisting of clinical grade fibrin hydrogel sealant containing rSDF-1 (0.1 μg per mL of hydrogel) showed >50% increase in cell viability following two weeks of in vitro culture, relative to the rSDF-1 untreated hydrogel control group. Based on this data, the biologic scaffold system described herein can be fused with rSDF-1 to increase CPC retention and cell viability.

Stromal cell-derived factor 1 (SDF-1) (SEQ ID NO: 1) amino acid sequence GenBank Accession Number: P48061.1 incorporated herein by reference.

1 mnakvvvvlv lvltalclsd gkpvslsyrc pcrffeshva ranvkhlkil ntpncalqiv
61 arlknnnrqv cidpklkwiq eylekalnkr fkm

Exemplary landmark residues, domains, and fragments of SDF-1 include, but are not limited to residues 1-21 (signal sequence), residues 22-93 (mature chain), residues 24-93 (mature chain), residues 24-88 (mature chain), residues 24-26 (helical region), residues 27-88 (chemokine_CXC domain), residues 31-34 (beta strand region), residues 36-38 (beta strand region), residues 39-93 (splicing variant), residues 41-43 (helical region), residues 53-55 (hydrogen bonded turn), or residues 89-93 (splicing variant). A fragment of a SDF-1 protein is less than the length of the full length protein, e.g., a fragment is at least 3, 4, 5, 6, 7, 8, 9, 10, 20, 30, 40, 50, 75, 80, 90 or 92 or more residues in length, but less than e.g., 93 residues in the case of SDF-1 above.

Fibrinogen (SEQ ID NO: 2) amino acid sequence GenBank Accession Number: CAA50740.1, incorporated herein by reference.

```
    1 mqngagasrt stiflngnre rplnvfcdme tdgggwlvfq rrmdgqtdfw rdwedyahgf
  61 gnisgefwlg nealhsltqa gdysirvdlr agdeavfaqy dsfhvdsaae yyrlhlegyh
121 gtagdsmsyh sgsvfsardr dpnsllisca vsyrgawwyr nchyanlngl ygstvdhqgv
181 swyhwkgfef svpftemklr prnfrspagg g
```

Thrombin (SEQ ID NO: 3) amino acid sequence GenBank Accession Number: NP_000497.1 incorporated herein by reference.

```
    1 mahvrglqlp gclalaalcs lvhsqhvfla pqqarsllqr vrrantflee vrkgnlerec
  61 veetcsyeea fealesstat dvfwakytac etartprdkl aaclegncae glgtnyrghv
121 nitrsgiecq lwrsryphkp einstthpga dlqenfcrnp dssttgpwcy ttdptvrrqe
181 csipvcgqdq vtvamtprse gssvnlsppl eqcvpdrgqq yqgrlavtth glpclawasa
241 qakalskhqd fnsavqlven fcrnpdgdee gvwcyvagkp gdfgycdlny ceeaveeetg
301 dgldedsdra iegrtatsey qtffnprtfg sgeadcglrp lfekksledk terellesyi
361 dgrivegsda eigmspwqvm lfrkspqell cgaslisdrw vltaahclly ppwdknften
421 dllvrigkhs rtryerniek ismlekiyih prynwrenld rdialmklkk pvafsdyihp
481 vclpdretaa sllqagykgr vtgwgnlket wtanvgkgqp svlqvvnlpi verpvckdst
541 riritdnmfc agykpdegkr gdacegdsgg pfvmkspfnn rwyqmgivsw gegcdrdgky
601 gfythvfrlk kwiqkvidqf ge
```

Exemplary landmark residues, domains, and fragments of thrombin include, but are not limited to residues 1-24 (signal sequence), residues 44-622 (mature protein), residues 44-198 (activation peptide fragment 1), residues 105-186 (Kringle domain), residues 199-327 (activation peptide fragment 2), residues 328-363 (thrombin light chain), residues 364-622 (thrombin heavy chain), residues 572-622 (thrombin-derived C-terminal peptide), or residues 551-573 (high affinity receptor-binding region—TP508 peptide). A fragment of a thrombin protein is less than the length of the full length protein, e.g., a fragment is at least 3, 4, 5, 6, 7, 8, 9, 10, 20, 30, 40, 50, 100, 200, 300, 400, 500 or more residues in length, but less than e.g., 622 residues in the case of thrombin above.

### EXAMPLE 4

### In Vivo Large Animal Data Demonstrated That Treatment of Meniscal Tears by Fibrin Hydrogel Encapsulated With CPCs and Infused With rSDF-1 Stimulated Soft Tissue Healing

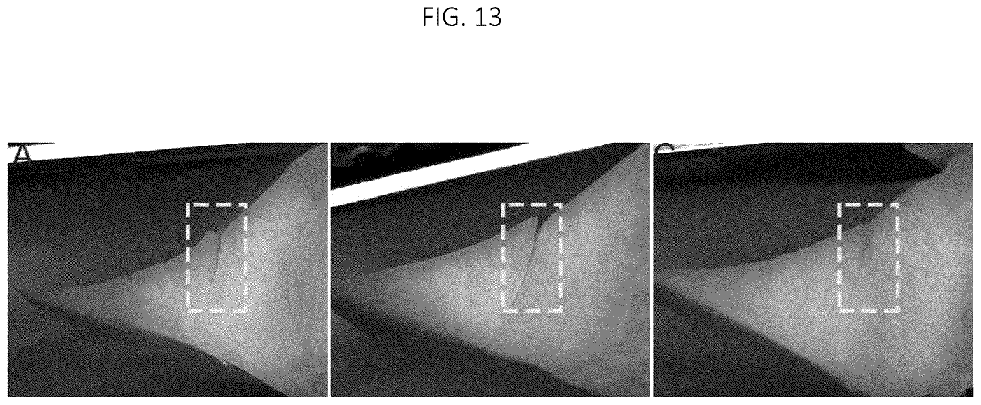

Medial parapatellar arthrotomy was performed on the right knees of young adult 13 to 18-month-old Yucatan mini-pigs. A longitudinal 1.0 cm tear was surgically created using a linear tissue punch in the central red-white zone of the anterior medial meniscus. There were three experimental groups: (1) Meniscus injury treated with suture repair alone (negative control group); (2) Meniscus injury that is suture repaired with fibrin gel only (gel alone control group); and (3) Meniscus injury that is suture repaired with CPC encapsulated fibrin gel (FIG. 13). A nonabsorbable 3-0 suture was used. Animals were kept for 12-weeks (3 months) before euthanasia for assessment of the meniscus to evaluate meniscal healing.

The scaffold was created by combining two components: component #1 including thrombin (350-700 units/mL), calcium chloride (30-50 μm/mL), SDF-1 (30 ng/mL), and cells in distilled water. Component #2 is Fibrinogen (60-120 mg/mL) and the synthetic crosslinker aprotinin (2250-3750 KIU/mL) in distilled water. No KGN was used in this experiment; and component #1 and #2 were combined in a 1:1 ratio by volume.

This data demonstrated that treatment of meniscal tears with the CPC encapsulated bioactive hydrogel achieved better tear reintegration and healing, compared to controls that were left untreated, or simply treated with the base hydrogel.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All references, e.g., U.S. patents, U.S. patent application publications, PCT patent applications designating the U.S., published foreign patents and patent applications cited herein are incorporated herein by reference in their entireties. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

While this invention has been particularly shown and described with references to preferred embodiments thereof, it will be understood by those skilled in the art that various changes in form and details may be made therein without departing from the scope of the invention encompassed by the appended claims.

What is claimed:

1-2. (canceled)

3. A method of repairing a musculoskeletal tissue defect or injury in a mammalian subject, the method comprising contacting the defect or a site of the injury with a bioactive scaffold comprising a chondrogenic fibrin glue into which a population of skeletal tissue derived mesenchymal progenitor cells (STMSCs) is encapsulated, wherein the chondrogenic fibrin glue comprises fibrinogen, thrombin, stromal cell derived factor 1 (SDF-1), and kartogenin (KGN): 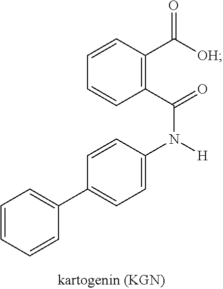 [SMILES: O=C(O)c1ccccc1C(=O)Nc1ccc(-c2ccccc2)cc1]
and
wherein the STMSCs comprise cartilage-derived mesenchymal progenitor cells (CPCs).

4. The method of claim 3, wherein said scaffold is administered to the subject by injection.

5. A method of manufacturing a bioactive scaffold composition comprising combining component 1 and component 2,
wherein component 1 comprises thrombin, calcium chloride, stromal cell derived growth factor 1 (SDF-1), kartogenin (KGN): 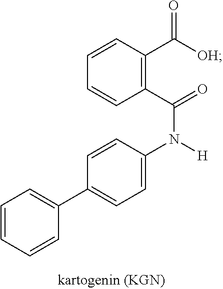 [SMILES: O=C(O)c1ccccc1C(=O)Nc1ccc(-c2ccccc2)cc1]
and cartilage-derived mesenchymal progenitor cells (CPCs),
wherein component 2 comprises fibrinogen and a synthetic crosslinker, and
wherein component 1 and component 2 are combined in a 1:1 ratio by volume.

6. The method of claim 5, wherein the thrombin is at a concentration from about 350-700 units/mL.

7. The method of claim 5, wherein the calcium chloride is at a concentration from about 30-50 μm/mL.

8. The method of claim 5, wherein the SDF-1 is at a concentration from about 1-50 ng/mL.

9. The method of claim 5, wherein the KGN is at a concentration from about 0.01-0.5 μg/mL.

10. The method of claim 5, wherein the fibrinogen is at a concentration of about 60-120 mg/mL.

11. The method of claim 5, wherein the synthetic crosslinker comprises aprotinin, and wherein the concentration of the crosslinker is about 2250-3750 KIU/mL.

12. The method of claim 5, wherein the CPCs are embedded from about 1.0×10⁴ to about 5.0×10⁵ cells/μL of the bioactive scaffold.

13. The method of claim 5, wherein the CPCs are embedded in a concentration of about 4.0×10⁵ cells per 10 μL of the bioactive scaffold.

14. The method of claim 5, wherein component 1 and component 2 are combined in a 1:1 ratio by volume.

15. A composition for repairing a musculoskeletal tissue defect or injury comprising a population of cartilage-derived mesenchymal progenitor cells (CPCs), wherein the CPCs express cell surface markers comprising CD166 CD54, or CD105, and wherein the CPCs do not express the cell surface markers comprising CD106, CD4, CD14, or CD34.

16. The composition of claim 15, wherein said CPCs comprise cells deposited with ATCC accession number PTA-127250.

17. The composition of claim 15, further comprising a bioactive scaffold composition comprising a population of cartilage-derived mesenchymal progenitor cells (CPCs), and further comprising fibrinogen, thrombin, stromal cell derived factor 1 (SDF-1), and kartogenin (KGN):

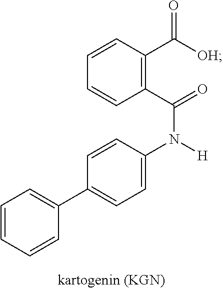 [SMILES: O=C(O)c1ccccc1C(=O)Nc1ccc(-c2ccccc2)cc1]

18. The composition of claim 15, wherein the CPCs in the population of cells are at least 50%, 60%, 75%, 80%, 90%, 95%, 98%, or 99% purified.

19. The composition of claim 15, wherein the CPCs expresses greater SOX9 relative to bone marrow-derived stromal cells (BMSCs), or wherein the CPCs express less COL10 relative to BMSCs.

20. (canceled)

21. The method of claim 3, wherein the fibrinogen comprises SEQ ID NO: 2 (GENBANK: CAA50740.1); the thrombin comprises SEQ ID NO: 3 (GENBANK: NP_000497.1); and the SDF-1 comprises SEQ ID NO: 1 (GENBANK: P48061.1).

22. The method of claim 5, wherein the fibrinogen comprises SEQ ID NO: 2 (GENBANK: CAA50740.1); the thrombin comprises SEQ ID NO: 3 (GENBANK: NP_000497.1); and the SDF-1 comprises SEQ ID NO: 1 (GENBANK: P48061.1).

23. The composition of claim 15, further comprising: fibrinogen comprising SEQ ID NO: 2 (GENBANK: CAA50740.1); thrombin comprising SEQ ID NO: 3 (GENBANK: NP_000497.1); and SDF-1 comprising SEQ ID NO: 1 (GENBANK: P48061.1).
